(19) United States
(12) Patent Application Publication
(10) Pub. No.: US 2006/0200247 A1
(43) Pub. Date: Sep. 7, 2006

(54) Implant and articular prosthesis comprising said implant

(76) Inventor: Olivier Charrois, Paris (FR)

Correspondence Address: YOUNG & THOMPSON, 745 SOUTH 23RD STREET, 2ND FLOOR, ARLINGTON, VA, 22202, US

(21) Appl. No.: 10/542,033

(22) PCT Filed: Jan. 12, 2004
(86) PCT No.: PCT/FR04/00044

§ 371 Date: Dec. 29, 2005

(51) Int. Cl. A61F 2/30; A61F 2/40; A61F 2/32

(52) U.S. Cl. 623/19.11; 623/22.17; 623/23.4

(57) ABSTRACT

An implant for an articular prosthesis comprising a first part (3) made of a polymer material defining a cavity (4) in which a second part (7) made of a ceramic material is accommodated, defining a cavity (8) which is essentially hemispherical in shape and which receives the head (2) of a second implant also comprising a rod (1), characterized in that the edge (10) of the second part (7) is buried in the first part (3) which is produced by duplicate moulding on the second part (7), also characterized in that the edge (9) of the first part (3) is provided with a chamfer (11′) against which the rod (1) of the second implant is thrust. An articular prosthesis comprising a first implant and a second implant comprising a rod (1) which is terminated by substantially spherical hed (2) which turns in a cavity (8) whose shape corresponds to that of the first implant, characterized in that the first implant is of the previous type.

The invention concerns the field of articular prostheses such as hip or shoulder prostheses.

It is customary to use hip prostheses formed, on the one hand, by a metal rod and a femoral head made of ceramic and of substantially spherical shape which the surgeon uses to replace the upper portion of the femur of the patient, and, on the other hand, by an acetabular implant which is intended to receive said femoral neck and which the surgeon implants, for example by sealing, in the patient's pelvis as a replacement for the natural acetabulum.

In a known preferred example of such a prosthesis, the acetabular implant is composed of a first part made of a polymer material, for example polyethylene, the general shape of which is that of a cup, and of a second part which is made of a ceramic material, for example aluminum, and which lines the inside of the first part. This second part defines a seat whose shape corresponds to that of the femoral head of the prosthesis. The inner edge of this seat is chamfered in such a way that it does not have a sharp angle and in such a way as to permit a given maximum inclination of the metal rod during the movements of the patient's thigh.

A problem posed by this configuration is that, when the metal rod assumes its maximum inclination, it strikes against the inner chamfered edge of the seat formed in the ceramic part. This represents a source of wear of the ceramic, leading to undesirable dispersion of particles in the region of the articulation, or even fracturing of the ceramic part affected by the impacts. Moreover, the rod then bears on the edge of the seat, and, if the relative movement of the thigh and of the pelvis seeks to continue, the normal geometry of the acetabular cups means there is a substantial risk of the femoral head escaping from its seat as a result of a “cam effect”, leading to luxation of the hip.

The object of the invention is to propose a novel acetabular implant design allowing the aforementioned problems to be remedied.

To this end, the subject of the invention is an implant for an articular prosthesis, of the type comprising a first part made of a polymer material and defining a cavity in which a second part made of a ceramic material is fitted, said second part defining a cavity of substantially hemispherical shape intended to receive a head of a second implant which also includes a rod, characterized in that the edge of said second part is recessed in the first part which is produced by duplicate molding on the second part, and in that the edge of said first part has a chamfer against which the rod of the second implant is intended to be able to abut.

Said first part preferably includes, in line with the upper limit of the cavity of the second part, a wall portion intended to form an abutment for the head during movements that are liable to cause luxation of the articulation.

The invention also relates to an articular prosthesis, of the type comprising a first implant and a second implant with a rod terminated by a head of substantially spherical shape which turns in a cavity of corresponding shape in the first implant, characterized in that said first implant is of the above type.

This articular prosthesis can in particular constitute a hip prosthesis or a shoulder prosthesis.

As will be understood, the invention consists in performing duplicate molding of the ceramic part of the first implant by a polymer material covering all of the upper edge of said ceramic part. The chamfer limiting the amplitude of the inclinations of the rod of the second implant is formed on the duplicate mold, such that the rod, which is generally made of metal, never comes into contact with a ceramic part. The only contacts that may exist in the articular prosthesis according to the invention are therefore ceramic-ceramic contacts and metal-polymer contacts. There is therefore no longer any metal-ceramic contact capable of causing substantial wear or fracturing of the ceramic. Moreover, the invention can lead to a modification of the geometry of the first implant, making luxation of the articulation more difficult.

In the preferred example of application of the invention to a hip prosthesis, the first implant is an acetabular implant, and the second implant replaces the terminal portion of the femur of the patient.

The invention will be better understood from reading the following description in which reference is made to the attached figures, in which:

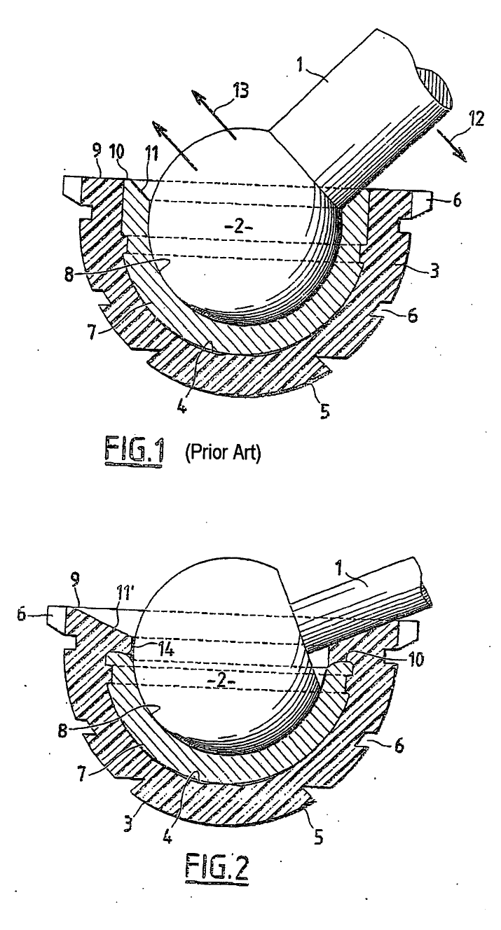

FIG. 1 is a schematic cross section through a hip prosthesis according to the prior art,

FIG. 2 is a schematic cross section through a hip prosthesis according to the invention, derived from the prosthesis in FIG. 1.

The hip prosthesis according to the prior art as shown in FIG. 1 includes the following elements:

an implant formed by a metallic rod 1 made of a biocompatible metal (stainless steel or titanium, for example) having, at its end, a femoral head 2 which is made of a ceramic such as aluminum and which is of a substantially spherical shape, this assembly of rod 1 and head 2 being intended to replace the terminal portion of the femur of the patient;

an acetabular implant which receives the femoral head 2, is intended to replace the acetabulum of the femoral articulation of the patient, and is composed of two parts:
a first part 3 made of a polymer material such as polyethylene, with an approximately hemispherical outer shape and provided with a cavity 4 defining a substantially spherical surface; this first part is intended to be implanted in the patient's pelvis, for example by sealing with the aid of a cement; to facilitate fixation in the pelvis, the outer surface 5 of the first part 3 includes various grooves and recesses 6;
a second part 7 made of a ceramic such as aluminum, lining the cavity 4 of the first part, and defining the seat 8 for the femoral head 2; for this purpose, this seat 8 has a spherical surface corresponding to the geometry of the femoral head 2.

It will be noted that the edges 9, 10 of the first 3 and second 7 parts directed toward the outside of the articulation are aligned in such a way as to define a single edge of the acetabular implant. The edge 10 of the second part 7 has a chamfer 11 on which, as is shown, the metal rod 1 bears at the maximum permitted clearance of the femur in the articulation (according to the arrow 12). As has been stated, this contact between metal and ceramic generates wear of the ceramic, even a risk of fracturing, in the area of the chamfer 11. Moreover, the chamfer 11 thus constitutes a bearing zone for the rod 1. If the movement of the rod 1 seeks to continue, this risks causing the head 2 to escape from the seat 8 in the direction of the arrows 13, in other words luxation of the patient's hip.

In the example of a hip prosthesis according to the invention shown in FIG. 2, the implant formed by the assembly of rod 1 and head 2 is once more present. The acetabular implant once again is made up of a first part 3 made of a polymer and with a general shape similar to that in FIG. 1, likewise with ribs and recesses 6 on its outer surface 5, and of a first part 7 made of ceramic and defining the seat 8 for the femoral head 2. The main difference from the configuration in the prior art shown in FIG. 1 its that here, according to the invention, the upper edge 9 of the first part 3 alone constitutes the upper edge of the acetabular implant. The upper edge 10 of the second part 7 is thus recessed inside the first part 3 which is produced by duplicate molding on the second part 7. The main consequence is that the chamfer 11′, which limits the clearance of the rod 1 and thus performs the same function as the chamfer 11 of the prior art, is no longer formed on the second part 7 made of ceramic, but instead on the upper edge 9 of the first part 3 made of polymer. The contacts between the rod 1 and the chamfer 11′ are therefore metal-polymer contacts which do not risk causing release of particle or pieces of ceramic into the patient's body.

The production of the first part 3 by duplicate molding on the second part 7 permits excellent complementarity between the two parts, and therefore an excellent hold of the second part 7 by the first part 3. This is crucial since the first part is fixed directly in the patient's pelvis.

Another advantage of the invention is that, as can be seen from FIGS. 1 and 2, while keeping similar dimensions for the main parts of the prosthesis (total size of the assembly, thickness of the various parts) it is possible to give the chamfer 11′ a greater inclination with respect to the vertical than in the prior art. The possible angle of clearance of the rod 1 is thus increased. This, together with the farther distance of the center of curvature of the movement of the rod from the edge of the seat for the head 2 when it bears on the chamfer 11′, makes it possible to reduce the risk of a cam effect being produced, as has been described above. Moreover, it is possible to form on the first part 3, in line with the upper limit of the cavity 8 of the second part 7, a wall portion 14 which continues said cavity 8 and which is intended to form an abutment for the head 2 when the rod 1 seeks to continue its movement after coming into abutment against the chamfer 11′. Luxation thus becomes more difficult.

It will be noted that the thickness of the polymer covering the edge 10 of the second part 7 must be at least 0.5 mm. It can of course be appreciably greater.

By way of example, a hip prosthesis according to the invention can have the following dimensions:

external dimension of the first part 3 made of polymer: 30×50 mm;

thickness of the first part 3 made of polymer: 6 mm;

thickness of the second part 7 made of ceramic: 4 mm;

radius of curvature of the seat 8 for the head 2: 14 mm;

inclination of the chamfer 11′ with respect to the vertical: 60°

height of the vertical wall portion 14: 4 mm.

The invention has been described and depicted in its application to a hip prosthesis. However, it can be applied to other types of articular prostheses having an analogous function, for example shoulder prostheses.

1. An implant for an articular prosthesis, of the type comprising a first part (3) made of a polymer material and defining a cavity (4) in which a second part (7) made of a ceramic material is fitted, said second part (7) defining a cavity (8) of substantially hemispherical shape intended to receive a head (2) of a second implant which also includes a rod (1), characterized in that the edge (10) of said second part (7) is recessed in the first part (3) which is produced by duplicate molding on the second part (7), and in that the edge (9) of said first part (3) has a chamfer (11′) against which the rod (1) of the second implant is intended to be able to abut.

2. The implant as claimed in claim 1, characterized in that said first part (3) comprises, in line with the upper limit of the cavity (8) of the second part (7), a wall portion (14) intended to form an abutment for the head (2) during movements that are liable to cause luxation of the articulation.

3. An articular prosthesis, of the type comprising a first implant and a second implant with a rod (1) terminated by a head (2) of substantially spherical shape which turns in a cavity (8) of corresponding shape in the first implant, characterized in that said first implant is of the type as claimed in claim 1.

4. The articular prosthesis as claimed in claim 3, characterized in that it constitutes a hip prosthesis, said first implant being an acetabular implant and said second implant replacing the terminal portion of the femur of the patient.

5. The articular prosthesis as claimed in claim 3, characterized in that it constitutes a shoulder prosthesis.

6. An articular prosthesis, of the type comprising a first implant and a second implant with a rod (1) terminated by a head (2) of substantially spherical shape which turns in a cavity (8) of corresponding shape in the first implant, characterized in that said first implant is of the type as claimed in claim 2.
